(19) United States
(12) Patent Application Publication
(10) Pub. No.: US 2009/0155805 A1
(43) Pub. Date: Jun. 18, 2009

(54) COPY NUMBER ALTERATIONS THAT PREDICT METASTATIC CAPABILITY OF HUMAN BREAST CANCER

(75) Inventors: Yi Zhang, San Diego, CA (US); Jack X. Yu, San Diego, CA (US); Yuqui Jiang, San Diego, CA (US); Yixin Wang, Basking Ridge, NJ (US)

Correspondence Address: HISCOCK & BARCLAY, LLP, 2000 HSBC PLAZA, 100 Chestnut Street, ROCHESTER, NY, 14604-2404, US

(73) Assignee: Veridex, LLC

(21) Appl. No.: 12/335,162

(22) Filed: Dec. 15, 2008

Related U.S. Application Data
(60) Provisional application No. 61/007,650, filed on Dec. 14, 2007.

(51) Int. Cl. C12Q 1/68

(52) U.S. Cl. 435/6

(57) ABSTRACT

Disclosed in this specification is a method of defining chromosome regions of prognostic value by summarizing the significance of all SNPs (single nucleotide polymorphism) in a predetermined section of a chromosome to define chromosome regions of prognostic value. Based on the SNPs in specified genes, a more accurate prognosis for breast cancer may be provided.

### CROSS-REFERENCE TO RELATED APPLICATIONS

This application claims priority to and the benefit of co-pending U.S. provisional patent application Ser. No. 61/007,650, filed Dec. 14, 2007, which application is incorporated herein by reference in its entirety.

### FIELD OF THE INVENTION

This invention relates, in one embodiment, to a method of providing a prognosis for breast cancer by determining the number of single nucleotide polymorphisms (SNPs) in specified genes.

### BACKGROUND OF THE INVENTION

Breast cancer is a heterogeneous disease that exhibits a wide variety of clinical presentations, histological types and growth rates. In patients with no detectable lymph node involvement (a population thought to be at low-risk) between 20-30% of the patients develop recurrent disease after five to ten years of follow-up. Identification of individuals in this group who are at risk for recurrence cannot be done reliably at present.

DNA copy number alterations (CNAs) or copy number polymorphisms (CNPs), such as deletions, insertion and amplifications, are believed to be one of the major genomic alterations that contribute to the carcinogenesis. Both conventional and array-based comparative genomic hybridizations have revealed chromosomal regions that are altered in breast tumors. There is no study, however, that used a high throughput, high resolution platform to investigate the relationship of DNA copy number alterations with breast cancer prognosis.

### SUMMARY OF THE INVENTION

The methods disclosed herein make it feasible to use copy number alterations (CNAs) to predict patient prognostic outcome. When combined with gene expression based signatures for prognosis, copy number signature (CNS) refines risk classification and can identify those breast cancer patients who have a significantly worse outlook in prognosis and a potential differential response to chemotherapeutic drugs.

In the examples discussed herein a high-throughput and high-resolution oligo-nucleotide based single nucleotide polymorphism (SNP) array technology was used to analyze the CNAs for more than 100,000 SNP loci in the breast cancer genome. In a large cohort of 313 LNN (lymph node negative) breast cancer patients CNAs were identified that were correlated with a subset of patients with a very high probability of developing distant metastasis. The prognostic power of the CNAs was validated in two independent patient cohorts. In addition, using published predictive gene signatures, the identified patient subgroups with different prognosis were tested for putative drug efficacy. The results indicate that combining DNA copy number analysis and gene expression analysis provides an additional and better means for risk assessment for breast cancer patients.

### BRIEF DESCRIPTION OF THE DRAWINGS

The present invention is disclosed with reference to the accompanying drawings, wherein:

FIG. 1 is an analysis workflow to identify the genes (SNPs) with prognostic copy number alterations (CNAs);

FIGS. 2A and 2B depict the chromosomal regions with prognostic CNAs;

FIG. 3 shows distant metastasis-free survival as a function of CNS;

FIG. 4 illustrations the sensitivity to chemotherapeutic compounds;

FIG. 5 graphically depicts the differentiation of ER-positive and ER-negative tumors; and

FIG. 6 illustrates certain data of ER-negative tumors.

The examples set out herein illustrate several embodiments of the invention but should not be construed as limiting the scope of the invention in any manner.

### DETAILED DESCRIPTION

Specific DNA copy number alterations (CNAs), such as deletions and amplifications, are major genomic alterations that contribute to the carcinogenesis and tumor progression through reduced apoptosis, unchecked proliferation, increased motility and angiogenesis. Because a significant proportion of genomic aberrations are unrelated to cancer biology and merely due to random neutral events, it is a challenge to identify those causative gene CNAs that are responsible for gene expression regulation that ultimately leads to malignant transformation and progression. Both fluorescence in situ hybridization and comparative genomic hybridizations (CGH) have revealed chromosomal regions that showed CNAs in breast tumors. In a recent study including 51 breast tumors, a high-resolution SNP array was used together with gene-expression profiling to refine breast cancer amplicon boundaries and narrow the list of potential driver genes. However, only a limited number of studies investigated the CNAs in relation to their prognostic significance while the sample sizes of these studies were too small to draw firm conclusions. In addition, fewer studies investigated breast cancer prognosis using combined analysis of CNAs and gene expression profiling with sufficient sample size and a technology that had appropriate coverage and mapping resolution of the human genome.

This specification describes the analysis of DNA copy numbers for over 100,000 SNP loci across the human genome in genomic DNA from 313 lymph node-negative (LNN) primary breast tumors for which genome-wide gene-expression data were also available. Combining these two data sets allowed the identification of genomic loci, and their mapped genes, that have high correlation with distance metastasis. The identified patient subgroups were further tested for putative drug efficacy based on published predictive signatures.

A combined analysis of DNA copy number and gene expression was performed on a large cohort of 313 LNN breast cancer patients who received no adjuvant systemic therapy. To our knowledge, this is the largest such study to analyze CNAs for breast cancer prognosis using the high-density SNP array technology that has much higher resolution than aCGH. A signature of 81 genes that showed CNAs and concordant gene expression regulation were identified from a training set of 200 LNN patients. This CNS was validated in the independent 113 LNN patients, as well as in an external aCGH data set of 116 LNN patients. Preliminary clinical utility has been demonstrated since the very poor prognostic group with a particularly rapid relapse identified by the 81-gene CNS actually constituted a subset of the poor prognostic patients predicted by the 76-gene GES alone. Thus by applying CNS in addition to GES, risk classification for breast cancer patients' prognosis is clearly improved. Furthermore, by using previously reported gene signature profiles for sensitivity to chemotherapeutic compounds, it was shown that this very poor prognostic group might be much more resistant to preoperative T/FAC combination chemotherapy, particularly against the cyclophosphamide and doxorubicin compounds, while benefiting from etoposide and topotecan. This may suggest that patients belonging to this category should be closely monitored and be managed with different chemotherapy regimes compared with other patient groups, and that the 81 genes of the CNS also play an important role in chemo sensitivity.

Previous studies investigating the association between gene amplification and breast cancer prognosis considered different breast cancer subtypes such as ER positive and ER negative as a single homogenous cohort. However, it is well known that these tumors are pathologically and biologically very different as evidenced by tremendous distinct global gene expression profiles. This dichotomy also extended to the global pattern of the DNA copy numbers. Therefore, the analysis needed to be performed separately for ER-positive and ER-negative (estrogen-receptor positive and negative) tumors. Indeed, the prognostic chromosomal regions identified from the ER-positive tumors share little in common with those from the ER-negative tumors. For example, chromosome region 8q is a widely known site of DNA amplification that is associated with poor prognosis in breast cancer. The region 8q was indeed a hotspot for amplification in ER-positive tumors, but contained no significant amplified areas for ER-negative tumors. Because ER-negative tumors constitute only a small percentage (˜25%) of the LNN breast cancers, it is reasonable to speculate that those studies that did not separate the two types of breast tumors in their analysis may had their conclusions overwhelmed by the results from the majority of the samples of ER-positive tumors. Another apparent difference between the two types of tumors observed from our analysis was at chromosome region 20q13.2-13.3. A gain in copy number of this region in ER-positive tumors, but by contrast, a loss in copy number of this region in ER-negative tumors, was related to an early recurrence. Taken together, these results re-emphasize that ER-positive and ER-negative tumors follow different biological pathways for cancer development and progression.

### Identification of Prognostic Chromosomal Regions

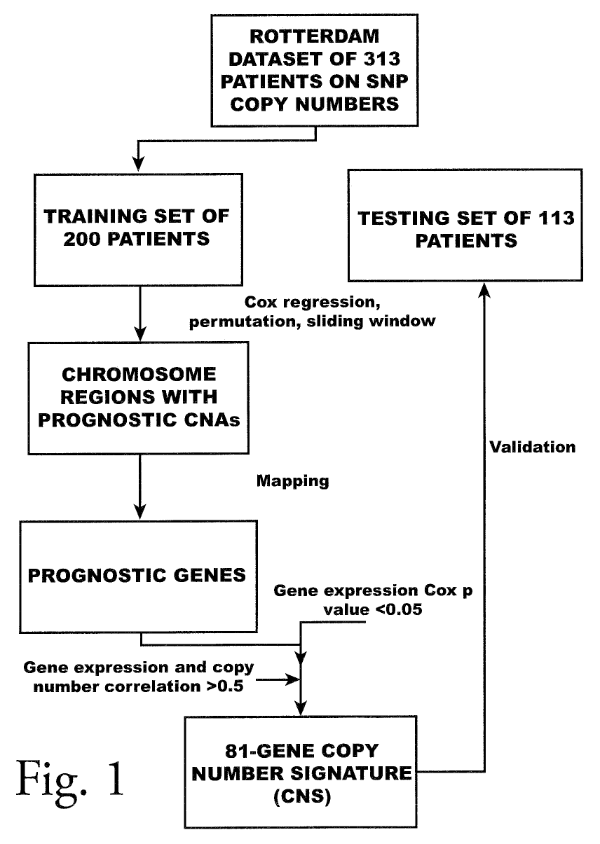

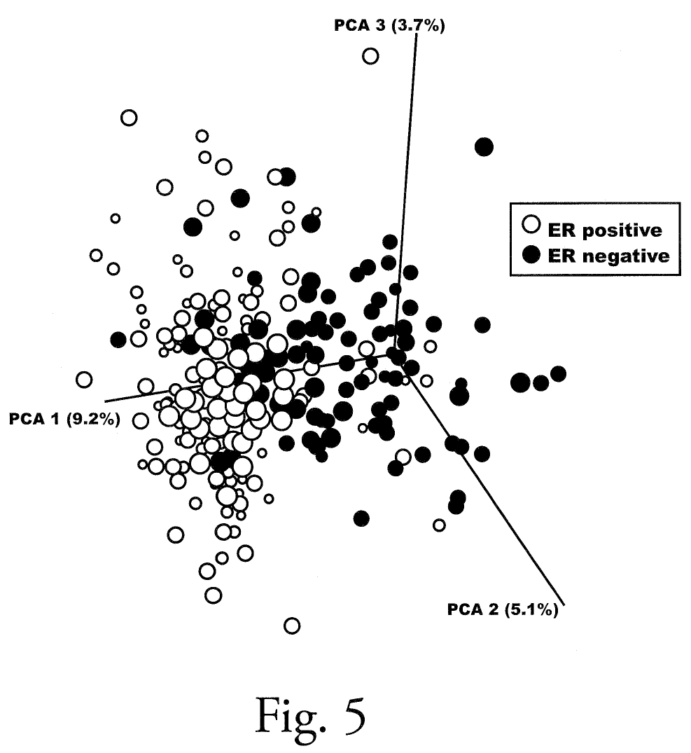

The median of the mean copy numbers computed from each SNP's interquartile copy number estimates was 2.1, consistent with the general assumption that the majority of the genome is diploid. Unsupervised analysis using PCA on all 313 tumors showed that chromosomal copy number variations displayed a clear trend of separation between ER-positive and ER-negative tumors (FIG. 5). Therefore, these two types of breast tumors not only differ on global gene expression profiles as indicated by many studies before, but also have distinct chromosomal variations on the DNA level. Therefore, it is necessary that the subsequent analysis be performed separately for ER-positive and ER-negative tumors. The patients were randomly divided into a training set of 200 patients (133 for ER-positive and 67 for ER-negative tumors) and a testing set of 113 patients (66 for ER-positive and 47 for ER negative tumors) (Table 1 and FIG. 1) in an approximate 2:1 ratio. The training set was used to identify prognostic chromosome regions and the mapped genes, and to construct a CNS to predict distance metastasis; the testing set was set aside solely for validation purpose.

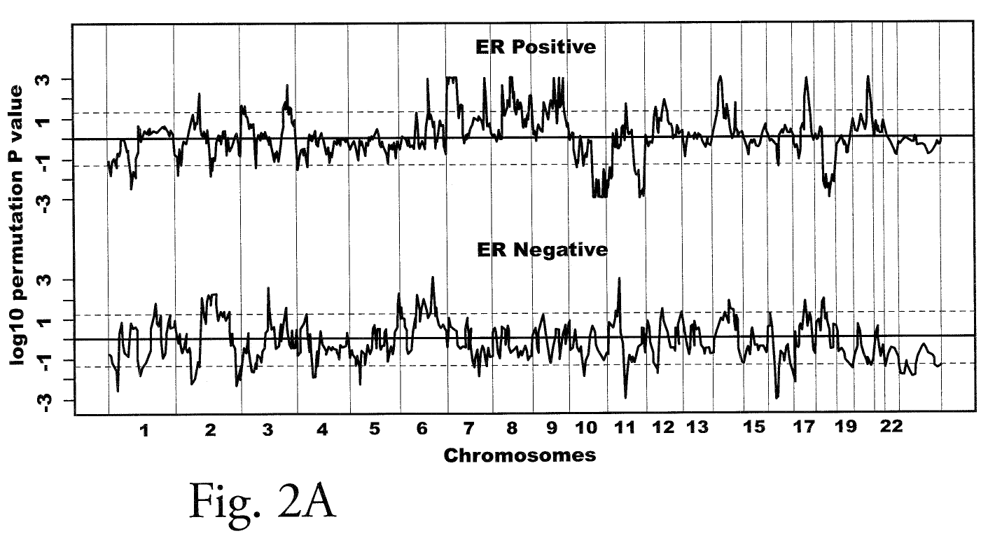

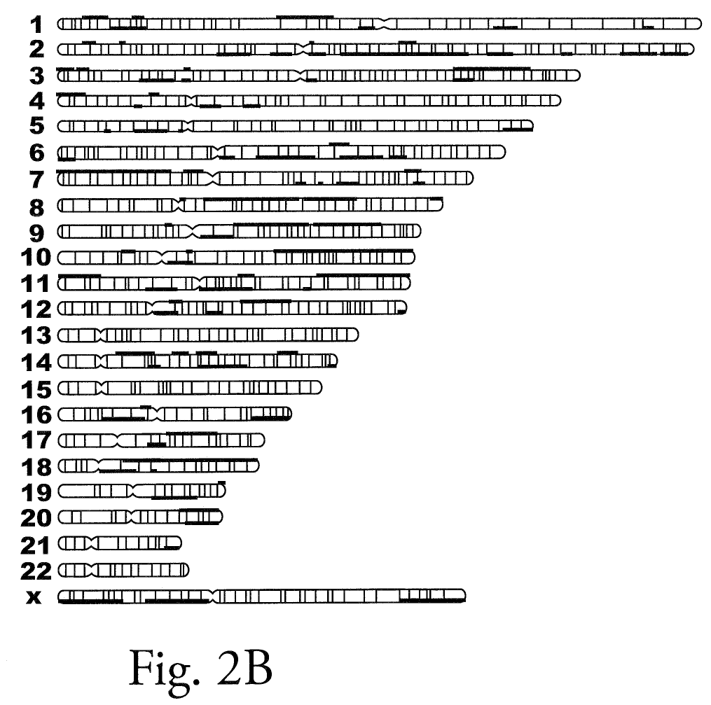

First, chromosome regions were identified whose CNAs were correlated with patients' DMFS. For ER-positive tumors, 45 chromosomal regions distributed over 17 chromosomes were identified as having CNAs that correlated with DMFS (FIG. 2A and Table 7), for ER-negative tumors there were 56 regions distributed over 19 chromosomes (Table 8). The total of these region sizes for ER-positive and ER-negative tumors were 521 (Table 4) and 496 Mb (Table 5), respectively. The prognostic chromosomal regions identified from the ER-positive tumors share little in common with those from the ER-negative tumors (FIGS. 2A and 2B).

In the training set of 200 patients an 81-gene prognostic copy number signature (CNS) was constructed that identified a subgroup of patients with a high probability of distant metastasis in the independent testing set of 113 patients (hazard ratio [HR]:2.8, 95% confidence interval [CI]:1.4-5.6,p=0.0036), and in an external data set of 116 patients (HR: 3.7, 95 CI: 1.3-10.6,p=0.0102). These high-risk patients constituted a subset of the high-risk patients predicted by our previously established 76-gene expression signature (GES). This very poor prognostic group identified by CNS and GES was putatively more resistant to preoperative paclitaxel and 5-FU-doxorubicin-cyclophosphamide (T/FAC) combination chemotherapy (p=0.0003), particularly against the doxorubicin and cyclophosphamide compound, while potentially benefiting from etoposide and topotecan.

### Patient Samples

Frozen tumor specimens of 313 LNN breast cancer patients selected from the tumor bank at the Erasmus Medical Center (Rotterdam, Netherlands) were used in this study. None of these patients did receive any systemic (neo)adjuvant therapy. The guidelines for local primary treatment were the same. Among these specimens, 273 were used to develop a 76-gene signature for the prediction of distant metastasis using Affymetrix U133A chips. The remaining 40 patients were used to study prognostic biological pathways. The study was approved by the Medical Ethics Committee of the Erasmus MC Rotterdam, The Netherlands (MEC 02.953), and was conducted in accordance to the Code of Conduct of the Federation of Medical Scientific Societies in the Netherlands (http://www.fmwv.nl/), and where ever possible the Reporting Recommendations for Tumor Marker Prognostic Studies REMARK was followed.

A sampling of 199 tumors were classified as ER positive and 114 as ER negative, using previously described ER (and PgR) cutoffs. Median age of patients at the time of surgery (breast conserving surgery: 230 patients; modified radical mastectomy: 83 patients) was 54 years (range, 26-83 years). The median follow-up time for surviving patients (n=220) was 99 months (range, 20-169 months). A total of 114 patients (36%) developed distant metastasis and were counted as failures in the analysis of DMFS. Of the 93 patients who died, 7 died without evidence of disease and were censored at last follow-up in the analysis of DMFS; 86 patients died after a previous relapse. The clinicopathological characteristics of the patients are given in Table 1. The data set containing the clinical and SNP data has been submitted to Gene Expression Omnibus database with accession number 10099 (http://www.ncbi.nlm.nih.gov/geo, username: jyu8; password: jackxyu).

The external array CGH (aCGH) data set of 116 LNN patients used in this study as an independent validation was downloaded from http://www.ncbi.nlm.nih.gov/geo/query/acc.cgi?acc=GSE8757. The clinical data (Table 1) related to this data set were kindly provided by Dr. Teschendorff, University of Cambridge, UK.

### DNA Isolation, Hybridization and DNA Copy Number Analysis

Genomic DNA was isolated from 5 to 10 30 μm tumor cryostat sections (10-25 mg) with QIAamp DNA mini kit (Qiagen, Venlo, Netherlands) according to the protocol provided by the manufacturer. Genomic DNA from each patient sample was allelo-typed using the Affymetrix GeneChip® Mapping 100K Array Set (Affymetrix, Santa Clara, Calif.) in accordance with the standard protocol. Briefly, 250 ng of genomic DNA was digested with either Hind III or XbaI, and then ligated to adapters that recognize the cohesive four base pair (bp) overhangs. A generic primer that recognizes the adapter sequence was used to amplify adapter-ligated DNA fragments with PCR conditions optimized to preferentially amplify fragments ranging from 250 to 2000 bp size using DNA Engine (MJ Research, Watertown, Mass.). After purification with the Qiagen MinElute 96 UF PCR purification system, a total of 40 μg of PCR product was fragmented and about 2.9 μg was visualized on a 4% TBE agarose gel to confirm that the average size of DNA fragments was smaller than 180 bp. The fragmented DNA was then labeled with biotin and hybridized to the Affymetrix GeneChip® Human Mapping 100K Array Set for 17 hours at 480 C in a hybridization oven. The arrays were washed and stained using Affymetrix Fluidics Station, and scanned with GeneChip Scanner 3000 G7 and GeneChip® Operating software (GCOS) (Affymetrix). GTYPE (Affymetrix) software was used to generate a SNP call for each probe set on the array. SNP call was determined for 96.6% of the probe sets across the study, with a standard deviation of 2.6%. CCNT 3.0 software was then used to generate a value representing the copy number of each probe set. This was done by comparing the hybridized intensities of each chip to a manufacturer provided reference set of intensity measurements for over 100 normal individuals of various ethnicities. The copy number measurements were then smoothed using the genomic smoothing function of CCNT with a window size of 0.5 Mb. The Affymetrix GeneChip@Human Mapping 100K Array Set contains 115,353 probe sets for which the exact mapping positions were defined. The median length of the interval between the probe sets was 8.6 kb, 75% of the intervals were less than 28 kb and 95% were less than 94.5 kb.

Identification of Chromosome Regions with Prognostic Copy Number Alterations

An integrated analytical method was designed to identify the chromosome regions and the mapped candidate genes whose CNAs were correlated with distance metastasis, by taking advantage of the availability of the genomic data on both RNA gene expression which were generated from our previous studies and DNA copy number from the same cohort of patients that became available in this study (FIG. 1). Our method is very similar in principle to the approach that Adler et al. took and described as stepwise linkage analysis of microarray signatures (SLAMS) to identify genetic regulators of expression signatures by intersecting genome-wide DNA copy number and gene expression data. ER-positive and ER-negative patients were analyzed separately and randomly split the patients, in an approximate 2:1 ratio, into a training set of 200 patients and a testing set of 113 patients (FIG. 1) while balancing on the clinical and pathological parameters including T stage, grade, menopausal status and recurrences. The training set was used to identify prognostic chromosome regions and the mapped genes, and to construct a CNS to predict distance metastasis; the testing set was set aside solely for validation purpose.

The first step in our analysis was to identify chromosome regions whose copy number alterations were correlated with distance metastasis. Briefly, in the training set the univariate Cox proportional-hazards regression was used to evaluate the statistical significance of the correlation between the copy number of each individual SNP and the time of DMFS. Then, to define prognostic chromosomal regions, chromosomes were scanned in steps of 1 Mb using a sliding window of 5 Mb which contained an average of 250 SNPs to compile the Cox regression p-values of all SNPs within the window and to determine a smoothed p-value of all these SNPs as a whole relative to permutated data sets. Briefly, for a given window of size 5 Mb containing n SNPs, let βᵢ and Pᵢ denote the Cox regression coefficient and the P value from the Cox regression for the i^{th} SNP, respectively. A log score S for this window was defined by summarizing the statistical significance of all SNPs within this window as a whole as follows:

$$\begin{matrix}S = {\sum }_{i = 1}^{n} - log ( {P}_{i} ) \cdot {I}_{i} \\ where \\ {I}_{i} = \{ \frac{1 if {β}_{i} > 0}{- 1 if {β}_{i} < 0}\end{matrix}$$

The indicator variable Iᵢ was used to account for and to distinguish the positively correlated copy number changes from the negatively correlated ones, indicated by the signs of the Cox regression coefficients βᵢ. The positive coefficients reflect that relapsing patients had higher copy numbers than disease-free patients and the negative coefficients suggested the opposite. To compute the smoothed p-values from the log scores, permutations were used to derive the null distribution of the log scores. Four hundred permutations were performed by shuffling the clinical information with regard to the patient IDs. From the smoothed p-values, the prognostic chromosomal regions were defined as the chromosomal segments within which the smoothed p-values were all less than 0.05.

### Construction of CNS and Predictive Model

Once the prognostic chromosome regions were identified, the well defined genes were mapped with an Entrez Gene ID within those regions using the UCSC Genome Browser (http://genome.ucsc.edu) Human March 2006 (hg18) assembly. Next, two filtering steps were used to select those genes with greater confidence of having prognostic values to build a CNS. First, those genes that have at least one corresponding Affymetrix U133A probe set ID were filtered down. Only those genes that had statistically significant Cox regression p-values (p<0.05) from the gene expression data were followed through. Second, the correlation between the gene expression levels and copy numbers must be greater than 0.5. If the gene contained multiple SNPs inside, then the SNP with the best Cox regression p-value was selected; if contained no SNP, then the nearest SNP was chosen. For U133A probe set, the one with the best Cox p-value was used.

To build a model using the genes in the CNS to predict distant metastasis, the genes numeric copy number estimates were transformed into discrete values, i.e., amplification, no change, or deletion. In order to do the transformation, the diploid copy numbers for each gene was estimated by performing a normal mixture modeling on the representative SNP's copy number data and using the main peak of the modeled distribution as the estimate of the diploid copy number. Then for amplification, it was defined as 1.5 units above the diploid copy number estimate to ensure low false positives due to the intrinsic data variability; whereas deletion was defined as 0.5 units below the diploid copy number estimate because of the nature of the alteration and the narrow distribution of the copy number data for copy number loss. Once the copy number data were transformed, the following simple and intuitive algorithm was used to build a predictive model. The algorithm classified a patient as a relapser if at least n genes had copy numbers altered in that patient, and as a non-relapser otherwise. All possible scenarios were examined for n ranging from 1 to all genes in the CNS and determined the value of n by examining the performance of the signature in the training set as measured by a significant log-rank test p-value and setting a lower limit for the percentage of positives (predicted relapsers) to avoid the situation of very small number of positives as n increases.

### Validation of CNS

The performance of the CNS was assessed both in the copy number data set of the remaining testing patients and in the external aCGH data set using the same algorithm described above. For the external data set, because it was derived from totally different aCGH technology and the data format was log 2 ratios, the cutoff for amplification was set at 0.45 while the cutoff for deletion was −0.35 to ensure comparable percentage of positives generated as the SNP array technology. As with the construction of the CNS, the validation was done in the ER positive and negative tumors separately using the corresponding subsets of genes in the CNS. The final performance shown, however, represented the combined performance for both ER positive and negative patients in the testing set.

### Putative Response to Chemotherapy

To test for putative responses of testing set patients to chemotherapeutic compounds, gene expression signatures in two published studies were used. The original gene expression data set and the R function for the prediction algorithm of diagonal linear discriminant analysis (DLDA) for the 30-gene preoperative paclitaxel, fluorouracil, doxorubicin and cyclophosphamide (T/FAC) response signature was downloaded from http://bioinformatics.mdanderson.org/pubdata.html. The model was trained from the original data set using the provided R function and then tested in our gene expression data set. For each of the seven gene expression signatures that predict sensitivity to individual chemotherapeutic drugs, the predicted probability of sensitivity to each compound using the Bayesian fitting of binary probit regression models was calculated with the help of Drs. Anil Potti and Joseph Nevins (for details see Potti A, Dressman H K, Bild A, Riedel R F, Chan G, Sayer R, et al. Genomic signatures to guide the use of chemotherapeutics. Nat Med. 2006 November; 12(11):1294-300).

### Statistical Analysis

Unsupervised analysis using principal component analysis (PCA) was performed on the copy number dataset with all SNPs to examine the potential subclasses of the tumors. Kaplan-Meier survival plots and log-rank tests were used to assess the differences in DMFS of the predicted high and low risk groups. Cox's proportional-hazard regression was performed to compute the HR and its 95% CI. Due to missing data on grade, multivariate Cox regression analysis was done by multiple imputation using Markov Chain Monte Carlo method under the general location model (Schafer J L. Analysis of incomplete multivariate data. London: Chapman & Hall/CRC Press; 1997). T tests were performed to assess the significance of differential therapeutic responses among the prognostic groups. All statistical analyses were performed using R version 2.6.2.

### Search for Prognostic Candidate Genes to Construct CNS

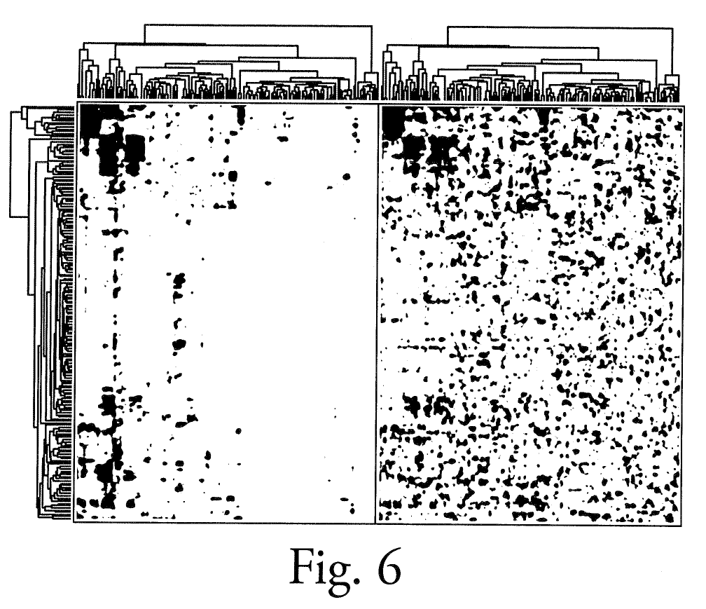

The gene expression profiling data from our previous studies of the same tumors were used (Wang Y, Klijn J G, Zhang Y, Sieuwerts A M, Look M P, Yang F, et al. Gene-expression profiles to predict distant metastasis of lymph-node-negative primary breast cancer. Lancet. 2005 Feb. 19;365(9460):671-9 and Yu J X, Sieuwerts A M, Zhang Y, Martens J W, Smid M, Klijn J G, et al. Pathway analysis of gene signatures predicting metastasis of node-negative primary breast cancer. BMC Cancer. 2007 Sep. 25;7(1):182) to screen for genes that had consistent change patterns between the gene expression profiles and the copy number variations. It was deemed reasonable that the change in copy numbers has to be reflected in the corresponding change in gene expression levels in order to have a phenotypic effect. Within these prognostic regions, a total of 2,833 and 3,656 genes were mapped for ER-positive tumors (Table 4) and ER-negative tumors (Table 5), respectively. For the ER-positive tumors, 122 genes had significant Cox regression p<0.05 in both the gene expression data and the copy number data, and showed the same direction for the changes in DNA copy number and gene expression. For the ER-negative tumors, 78 genes had significant p-values in both data sets, and showed the same direction of alterations (FIG. 6). Of these, 53 (43%) genes for ER-positive and 28 (36%) genes for ER-negative tumors, respectively, had correlation coefficients between gene expression and copy number greater than 0.5. Thus in total 81 prognostic candidate genes were identified which were then used as CNS for prognosis (Table 2 and Table 6A and 6B).

### Validation of CNS

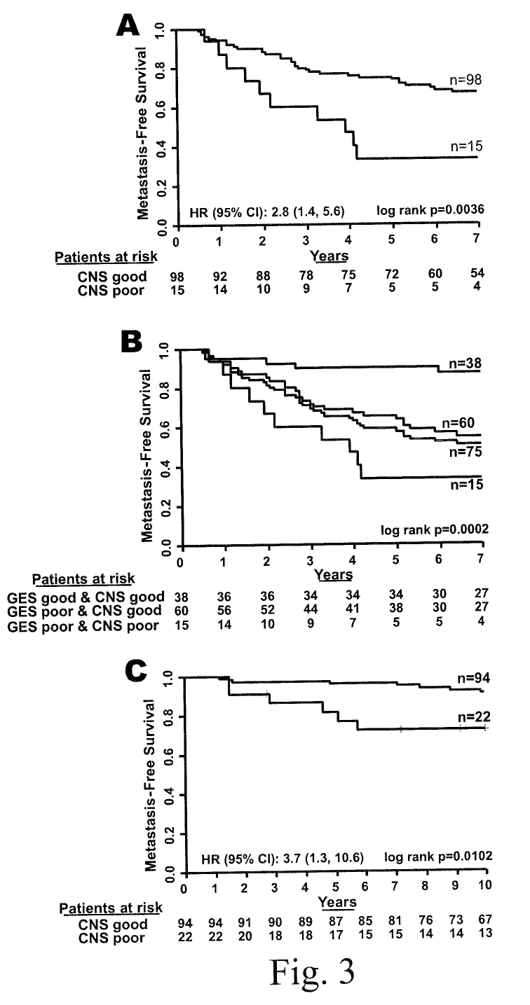

The validation was done in the ER positive and negative tumors separately for the testing set using 53 and 28 genes from the CNS, respectively. The final performance shown represented the combined results of the 2 subgroups. In the testing set of 113 independent patients, the Kaplan-Meier analyses of the two patient groups stratified by the 81-gene CNS showed a statistically significant difference in time to distance metastasis (FIG. 3, A) with a hazard ratio (HR) of 2.8 (p=0.0036). The estimated rate of distance metastasis at 5 years for the two groups was 27% [95% confidence interval (CI), 17% to 35%] and 67% (95% CI, 32% to 84%), respectively. When used in conjunction with our previously identified 76-gene GES (Wang Y, Klijn J G, Zhang Y, Sieuwerts A M, Look M P, Yang F, et al. Gene-expression profiles to predict distant metastasis of lymph-node-negative primary breast cancer. Lancet. 2005 Feb. 19;365(9460):671-9), the patient group with worse prognosis outcome defined by the 81-gene CNS remained the same with 67% of estimated distance metastasis at 5 years. The 76-gene GES stratified the other patient group with better prognosis further to a good and a poor prognosis group with the 5-year estimated rate of recurrence at 11% and 37%, respectively (FIG. 3, B). This result led to three prognostic groups, which were defined as good, poor and very poor groups for GES good/CNS good, GES poor/CNS good, GES poor/CNS poor groups, respectively. Multivariate Cox regression analysis of both signatures together with traditional clinical and pathological factors showed that the combination of the two signatures was the only significant (likelihood ratio test p=0.0003) prognostic factor for DMFS, with HRs of 8.86 comparing the very poor versus good prognostic group, and 3.59 for comparison of the poor versus the good prognostic group (Table 3).

Next, the CNS were tested in a completely independent external data set of 116 LNN patients (79 ER-positive and 37 ER-negative tumors) derived from a lower resolution aCGH technology (Chin S F, Teschendorff A E, Marioni J C, Wang Y, Barbosa-Morais N L, Thorne N P, et al. High-resolution array-CGH and expression profiling identifies a novel genomic subtype of ER negative breast cancer. Genome Biol. 2007 Oct. 9;8(10):R215). The 81-gene CNS significantly stratified this patient cohort (FIG. 3, C) into two prognostic groups with a HR of 3.7 (p=0.0102) and remained to be the only significant prognosticator in a multivariate Cox regression analysis including age, tumor size, grade, ER status (p=0.015). The lower rate of distance metastasis at 5 years (19%) for the poor prognostic group, compared with that of our own data set, was likely due to the smaller tumor sizes (78% smaller than 2 cm) and the fact that over one-third of the patients had received adjuvant hormone and/or chemotherapy in this cohort (Table 1).

### Response to Chemotherapy

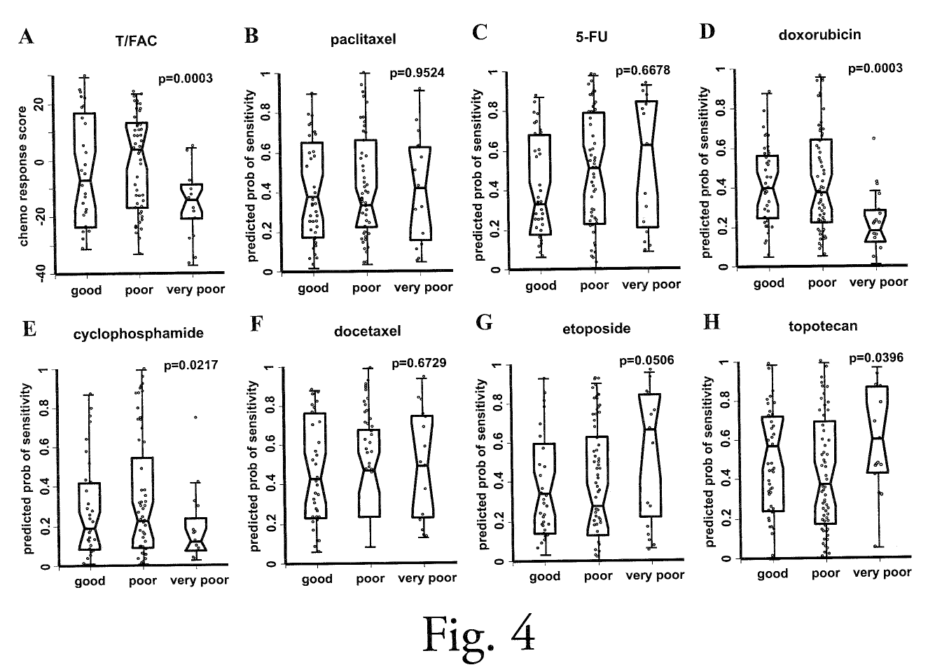

The chemotherapy response profiles were subsequently investigated for the three prognostic groups determined by the GES and CNS prognostic assays using well-validated gene signatures derived from two studies (Potti A, Dressman H K, Bild A, Riedel R F, Chan G, Sayer R, et al. Genomic signatures to guide the use of chemotherapeutics. Nat Med. 2006 Nov.;12(11):1294-300 and Hess K R, Anderson K, Symmans W F, Valero V, Ibrahim N, Mejia J A, et al. Pharmacogenomic predictor of sensitivity to preoperative chemotherapy with paclitaxel and fluorouracil, doxorubicin, and cyclophosphamide in breast cancer. J Clin Oncol. 2006 Sep. 10;24(26):4236-44) for which follow-up validation studies were also available (Bonnefoi H, Potti A, Delorenzi M, Mauriac L, Campone M, Tubiana-Hulin M, et al. Validation of gene signatures that predict the response of breast cancer to neoadjuvant chemotherapy: a substudy of the EORTC 10994/BIG 00-01 clinical trial. Lancet Oncol. 2007 Dec.;8(12):1071-8 and Peintinger F, Anderson K, Mazouni C, Kuerer H M, Hatzis C, Lin F, et al. Thirty-gene pharmacogenomic test correlates with residual cancer burden after preoperative chemotherapy for breast cancer. Clin Cancer Res. 2007 Jul. 15;13(14):4078-82). Firstly, using a previously published 30-gene signature that predicted pathological complete response (pCR) to preoperative T/FAC chemotherapy (Hess K R, Anderson K, Symmans W F, Valero V, Ibrahim N, Mejia J A, et al. Pharmacogenomic predictor of sensitivity to preoperative chemotherapy with paclitaxel and fluorouracil, doxorubicin, and cyclophosphamide in breast cancer. J Clin Oncol. 2006 Sep. 10;24(26):4236-44), each patient in the different prognostic subgroups was assigned into 2 response groups: either as having pCR or still with residual disease. Only 2 of the 15 patients (13%) in the very poor prognostic group were predicted as having pCR, while 34 of the 60 patients (57%) and 14 of the 38 patients (37%) in the poor and good prognostic groups, respectively, were predicted as having pCR. The chemo response score for the very poor prognostic group was significantly lower than those of the poor prognostic group (p=0.0003), indicating that these patients would be much more resistant to preoperative T/FAC chemotherapy in case these patients would have received pre-operative T/FAC chemotherapy (FIG. 4, A). Secondly, response profiles were determined for the three prognostic groups against seven individual chemotherapeutic compounds using expression signatures established on cell lines (Potti A, Dressman H K, Bild A, Riedel R F, Chan G, Sayer R, et al. Genomic signatures to guide the use of chemotherapeutics. Nat Med. 2006 Nov.;12(11):1294-300). For each compound, the predicted probability of sensitivity to the compound was calculated using the Bayesian fitting of binary probit regression models. Compared with the poor prognostic group, the patients in the very poor prognostic group appeared to be more resistant to doxorubicin (FIG. 4, D) and cyclophosphamide (FIG. 4, E), consistent with the prediction of response to T/FAC by the 30-gene signature (FIG. 4, A). On the other hand, the very poor prognosis group was more sensitive to etoposide (FIG. 4, G) and topotecan (FIG. 4, H). Thus, when combined with gene expression based signatures for prognosis and therapy prediction, CNAs measured by SNP arrays improve risk classification and can identify those breast cancer patients who have a significantly worse outlook in prognosis and a potential differential response to chemotherapeutic drugs.

TABLE 1
Clinical and pathological characteristics of patients and their tumors

| | All patients | | | | Validation set | | | |
|---|---|---|---|---|---|---|---|---|
| Characteristics | (n = 313) | | Training set (n = 200) | | (n = 113) | | External validation set (n = 116) | |
| Age, years | | | | | | | | |
| Mean (SD) | 54 | (12) | 54 | (12) | 54 | (12) | 57 | (10) |
| <=40 | 45 | (14%) | 30 | (15%) | 15 | (13%) | 6 | (5%) |
| 41-55 | 134 | (43%) | 84 | (42%) | 50 | (44%) | 41 | (35%) |
| 56-70 | 98 | (31%) | 62 | (31%) | 36 | (32%) | 68 | (59%) |
| >70 | 36 | (12%) | 24 | (12%) | 12 | (11%) | 1 | (1%) |
| Menopausal status | | | | | | | | |
| Premenopausal | 152 | (49%) | 96 | (48%) | 56 | (50%) | 38 | (33%) |
| Postmenopausal | 161 | (51%) | 104 | (52%) | 57 | (50%) | 78 | (67%) |
| T stage | | | | | | | | |
| T1 | 153 | (49%) | 97 | (49%) | 56 | (49%) | 90 | (78%) |
| T2 | 148 | (47%) | 95 | (47%) | 53 | (47%) | 26 | (22%) |
| T3/4 | 11 | (4%) | 8 | (4%) | 3 | (3%) | 0 | |
| Unknown | 1 | (0%) | 0 | | 1 | (1%) | 0 | |
| Grade | | | | | | | | |
| Poor | 165 | (53%) | 111 | (56%) | 54 | (48%) | (48%) | (42%) |
| Moderate | 45 | (14%) | 29 | (14%) | 16 | (14%) | (34%) | (29%) |
| Good | 6 | (2%) | 3 | (2%) | 3 | (3%) | (34%) | (29%) |
| Unknown | 97 | (31%) | 57 | (28%) | 40 | (35%) | 0 | |
| ER status | | | | | | | | |
| Positive | 199 | (64%) | 133 | (67) | 66 | (58%) | (79%) | (68%) |
| Negative | 114 | (36%) | 67 | (33) | 47 | (42%) | (37%) | (32%) |
| PR status | | | | | | | | |
| Positive | 156 | (50%) | 100 | (50%) | 56 | (50%) | NA | |
| Negative | 148 | (47%) | 92 | (46%) | 56 | (50%) | NA | |
| Unknown | 9 | (3%) | 8 | (4%) | 1 | (1%) | NA | |
| Metastasis within 5 years | | | | | | | | |
| Yes | 99 | (32%) | 64 | (32%) | 35 | (31%) | 8 | (7%) |
| No | 204 | (65%) | 127 | (64%) | 77 | (68%) | 104 | (90%) |
| Censored | 10 | (3%) | 9 | (4%) | 1 | (1%) | 4 | (3%) |
| Adjuvant systemic | | | | | | | | |
| therapy | | | | | | | | |
| Yes | 0 | | 0 | | 0 | | 43 | (37%) |
| No | 313 | (100%) | 200 | (100%) | 113 | (100%) | 71 | (61%) |

| | | | | | |
|---|---|---|---|---|---|
| Unknown | 0 | 0 | 0 | 2 | (2%) |

Grade was assessed by regional pathologists and reflects the current practice during the years the tumors were collected; ER positive and PgR positive: >10 fmol/mg protein or >10% positive tumor cells. NA, not available.

TABLE 2
Description of the 81 genes used as the copy number signature (CNS)

| | Prognostic genes with copy number alteration |
|---|---|
| Gain in ER+ tumors | SMC4, PDCD10, PREP, CBX3, NUP205, TCEB1, TERF1, TPD52, GGH, TRAM1, |
| | ZBTB10, YTHDF3, EIF3E, POLR2K, RPL30, CCNE2, RAD54B, MTERFD1, ENY2, |
| | DPY19L4, ZNF623, SCRIB, SLC39A4, ATP6V1G1, PSMA6, STRN3, CLTC, TRIM37, |
| | NME1, NME2, RPS6KB1, PPM1D, MED13, SLC35B1, APPBP2, MKS1, C17orf71, |
| | HEATR6, TMEM49, USP32, ANKRD40, NME1-NME2, ZNF264, ZNF304, ATP5E, |
| | CSTF1, PPP1R3D, AURKA, RAE1, STX16, C20orf43, RAB22A |
| Loss in ER+ tumors | TCTN3 |
| Gain in ER− tumors | C1orf9, COX5B, EIF5B, DDX18, TSN, p20, METTL5, MGAT1, TUBB2A, RWDD1, |
| | PGM3, FOXO3, CDC40, REV3L, HDAC2, TSPYL4, C6orf60, ASF1A, MED23, |
| | TSPYL1, ACTR10, KIAA0247, RARA, KRT10, RIOK3, IMPACT |
| Loss in ER− tumors | HDAC1, BSDC1 |

TABLE 3
Multivariate Cox regression analysis
of the GES and CNS combination

| | Multivariate analysis | | |
|---|---|---|---|
| | HR | (95% CI) | p |
| Age (per 10-yr increment) | 0.77 | (0.48-1.22) | 0.2573 |
| Post versus premenopausal | 1.34 | (0.45-3.97) | 0.5920 |
| Grade 1 and 2 versus 3 | 0.45 | (0.17-1.19) | 0.1060 |
| Tumor size >20 mm vs ≦20 mm | 1.02 | (0.54-1.92) | 0.9583 |
| ER negative versus positive | 1.07 | (0.52-2.19) | 0.8590 |
| GES & CNS combination | | | |
| poor versus good | 3.59 | (1.35-9.49) | 0.0102 |
| very poor versus good | 8.86 | (2.76-28.4) | 0.0002 |

HR = hazard ratio; 95% CI = 95% confidence interval.

TABLE 4
Chromosome regions with prognostic copy number
alterations (CNAs) for ER-positive tumors

| | Chromosome | No. | Total region size | Total No. | | No. SNPs within |
|---|---|---|---|---|---|---|
| Chromosome | size (Mb) | regions | (Mb) | SNPs | No. genes | genes |
| 1 | 245.12 | 3 | 32.64 | 1257 | 224 | 440 |
| 2 | 242.40 | 4 | 12.18 | 391 | 69 | 142 |
| 3 | 198.70 | 5 | 38 | 1791 | 183 | 786 |
| 4 | 191.09 | 2 | 13.67 | 408 | 106 | 141 |
| 5 | 180.61 | 0 | 0 | 0 | 0 | 0 |
| 6 | 170.82 | 1 | 6.23 | 255 | 37 | 128 |
| 7 | 158.62 | 3 | 55.75 | 3212 | 237 | 1294 |
| 8 | 146.05 | 5 | 58.6 | 2629 | 264 | 938 |
| 9 | 138.17 | 3 | 52.57 | 2178 | 227 | 726 |
| 10 | 135.23 | 4 | 57.82 | 2434 | 342 | 1000 |
| 11 | 134.17 | 3 | 55.27 | 2100 | 444 | 825 |
| 12 | 132.29 | 3 | 20.98 | 959 | 58 | 340 |
| 13 | 114.05 | 0 | 0 | 0 | 0 | 0 |
| 14 | 106.31 | 4 | 32.5 | 1747 | 172 | 607 |
| 15 | 100.18 | 0 | 0 | 0 | 0 | 0 |
| 16 | 88.37 | 1 | 1.82 | 4 | 2 | 1 |
| 17 | 78.18 | 1 | 17.64 | 558 | 180 | 201 |
| 18 | 76.07 | 1 | 49.73 | 2622 | 145 | 760 |
| 19 | 63.46 | 1 | 2.25 | 27 | 57 | 17 |
| 20 | 62.38 | 1 | 13.14 | 441 | 86 | 150 |
| 21 | 46.92 | 0 | 0 | 0 | 0 | 0 |
| 22 | 48.98 | 0 | 0 | 0 | 0 | 0 |
| X | 154.41 | 0 | 0 | 0 | 0 | 0 |
| Total | 3012.60 | 45 | 521 | 23013 | 2833 | 8496 |

TABLE 5
Chromosome regions with prognostic copy number
alterations (CNAs) for ER-negative tumors

| | Chromosome | No. | Total region size | Total No. | | No. SNPs within |
|---|---|---|---|---|---|---|
| Chromosome | size (Mb) | regions | (Mb) | SNPs | No. genes | genes |
| 1 | 245.12 | 4 | 27.91 | 880 | 278 | 460 |
| 2 | 242.40 | 9 | 106.87 | 4185 | 555 | 1459 |
| 3 | 198.70 | 4 | 23.92 | 728 | 189 | 248 |
| 4 | 191.09 | 3 | 13.67 | 657 | 66 | 207 |
| 5 | 180.61 | 5 | 21.71 | 855 | 127 | 337 |
| 6 | 170.82 | 5 | 50.78 | 2679 | 193 | 891 |
| 7 | 158.62 | 4 | 14.35 | 613 | 107 | 310 |
| 8 | 146.05 | 0 | 0 | 0 | 0 | 0 |
| 9 | 138.17 | 1 | 10.62 | 0 | 1 | 0 |
| 10 | 135.23 | 1 | 8.83 | 200 | 48 | 85 |
| 11 | 134.17 | 3 | 31.25 | 977 | 466 | 349 |
| 12 | 132.29 | 3 | 14.19 | 651 | 41 | 238 |
| 13 | 114.05 | 0 | 0 | 0 | 0 | 0 |
| 14 | 106.31 | 3 | 22.1 | 970 | 146 | 501 |
| 15 | 100.18 | 0 | 0 | 0 | 0 | 0 |
| 16 | 88.37 | 2 | 28.22 | 896 | 265 | 470 |
| 17 | 78.18 | 1 | 5.88 | 99 | 182 | 28 |
| 18 | 76.07 | 2 | 13.15 | 611 | 45 | 163 |
| 19 | 63.46 | 1 | 15.77 | 209 | 360 | 107 |
| 20 | 62.38 | 1 | 12.41 | 423 | 85 | 143 |
| 21 | 46.92 | 1 | 3.63 | 76 | 66 | 44 |
| 22 | 48.98 | 0 | 0 | 0 | 0 | 0 |
| X | 154.41 | 3 | 70.44 | 1118 | 436 | 300 |
| Total | 3012.60 | 56 | 496 | 16827 | 3656 | 6340 |

TABLE 6A
Description of the 81 genes used as the CNS

| | | | | | 100K | |
|---|---|---|---|---|---|---|
| | | | | U133A | Array | |
| gene | chromosome | Entrez | | Cox P | SNP ID | SNP Cox |
| symbol | location | ID | U133A ID | value | (SNP_A-) | P value |
| SMC4 | 3q26.1 | 10051 | 201664_at | 0.0001 | 1706664 | 0.0001 |
| PDCD10 | 3q26.1 | 11235 | 210907_s_at | 0.0101 | 1753577 | 0.0115 |
| PREP | 6q22 | 5550 | 204117_at | 0.0288 | 1692699 | 0.0116 |
| CBX3 | 7p15.2 | 11335 | 201091_s_at | 0.0058 | 1674739 | 0.0003 |
| NUP205 | 7q33 | 23165 | 212247_at | 0.0093 | 1657909 | 0.0004 |
| TCEB1 | 8q21.11 | 6921 | 202823_at | 0.0153 | 1684065 | 0.0079 |
| TERF1 | 8q13 | 7013 | 203448_s_at | 0.042 | 1745614 | 0.0061 |
| TPD52 | 8q21 | 7163 | 201690_s_at | 0.0048 | 1665579 | 0.019 |
| GGH | 8q12.3 | 8836 | 203560_at | 0.0215 | 1682989 | 0.0143 |
| TRAM1 | 8q13.3 | 23471 | 201398_s_at | 0.0066 | 1695245 | 0.0133 |
| ZBTB10 | 8q13-q21.1 | 65986 | 219312_s_at | 0.0003 | 1656394 | 0.005 |
| YTHDF3 | 8q12.3 | 253943 | 221749_at | 0.0056 | 1719283 | 0.009 |
| EIF3E | 8q22-q23 | 3646 | 208697_s_at | 0.0306 | 1689974 | 0.0149 |
| POLR2K | 8q22.2 | 5440 | 202634_at | 0.037 | 1642344 | 0.0235 |
| RPL30 | 8q22 | 6156 | 200062_s_at | 0.0498 | 1747204 | 0.0185 |
| CCNE2 | 8q22.1 | 9134 | 205034_at | 0.0013 | 1659515 | 0.028 |
| RAD54B | 8q21.3-q22 | 25788 | 219494_at | 0.019 | 1663487 | 0.0354 |
| MTERFD1 | 8q22.1 | 51001 | 219363_s_at | 0.0291 | 1717843 | 0.0174 |
| ENY2 | 8q23.1 | 56943 | 218482_at | 0.0128 | 1675508 | 0.0088 |
| DPY19L4 | 8q22.1 | 286148 | 213391_at | 0.0001 | 1727257 | 0.0091 |
| ZNF623 | 8q24.3 | 9831 | 206188_at | 0.0005 | 1695955 | 0.0121 |
| SCRIB | 8q24.3 | 23513 | 212556_at | 0.0323 | 1695955 | 0.0121 |
| SLC39A4 | 8q24.3 | 55630 | 219215_s_at | 0.0056 | 1695955 | 0.0121 |
| ATP6V1G1 | 9q32 | 9550 | 208737_at | 0.0499 | 1712044 | 0.0066 |
| TCTN3 | 10q23.33 | 26123 | 212123_at | −0.03 | 1647197 | −0.0179 |
| PSMA6 | 14q13 | 5687 | 208805_at | 0.0053 | 1739239 | 0.0265 |
| STRN3 | 14q13-q21 | 29966 | 204496_at | 0.002 | 1657718 | 0.0021 |
| CLTC | 17q11-qter | 1213 | 200614_at | 0.0011 | 1665731 | 0.0096 |
| TRIM37 | 17q23.2 | 4591 | 213009_s_at | 0.0036 | 1740610 | 0.0025 |
| NME1 | 17q21.3 | 4830 | 201577_at | 0.0478 | 1735518 | 0.0006 |
| NME2 | 17q21.3 | 4831 | 201268_at | 0.0422 | 1665752 | 0.0002 |
| RPS6KB1 | 17q23.1 | 6198 | 204171_at | 0.0002 | 1665339 | 0.0028 |
| PPM1D | 17q23.2 | 8493 | 204566_at | 0.0015 | 1738127 | 0.0035 |
| MED13 | 17q22-q23 | 9969 | 201987_at | 0.0001 | 1758346 | 0.0042 |
| SLC35B1 | 17q21.33 | 10237 | 202433_at | 0.0356 | 1722156 | 0.003 |
| APPBP2 | 17q21-q23 | 10513 | 202630_at | 0.0117 | 1707055 | 0.0045 |
| MKS1 | 17q22 | 54903 | 218630_at | 0.0272 | 1704909 | 0.0343 |
| C17orf71 | 17q22 | 55181 | 218514_at | 0.0069 | 1740610 | 0.0025 |
| HEATR6 | 17q23.1 | 63897 | 218991_at | 0.0026 | 1687894 | 0.0014 |
| TMEM49 | 17q23.1 | 81671 | 220990_s_at | 0.0044 | 1668378 | 0.0071 |
| USP32 | 17q23.2 | 84669 | 211702_s_at | 0.0042 | 1674736 | 0.0026 |
| ANKRD40 | 17q21.33 | 91369 | 211717_at | 0.0468 | 1744474 | 0.046 |
| NME1- | 17q21.3 | 654364 | 201268_at | 0.0422 | 1735518 | 0.0006 |
| NME2 | | | | | | |
| ZNF264 | 19q13.4 | 9422 | 205917_at | 0.0068 | 1706627 | 0.0078 |
| ZNF304 | 19q13.4 | 57343 | 207753_at | 0.0331 | 1645690 | 0.0129 |
| ATP5E | 20q13.32 | 514 | 217801_at | 0.0118 | 1693246 | 0.0126 |
| CSTF1 | 20q13.31 | 1477 | 32723_at | 0.0054 | 1656558 | 0.0093 |
| PPP1R3D | 20q13.3 | 5509 | 204554_at | 0.0205 | 1700634 | 0.0249 |
| AURKA | 20q13.2-q13.3 | 6790 | 204092_s_at | 0.0001 | 1739857 | 0.0093 |
| RAE1 | 20q13.31 | 8480 | 201558_at | 0.0032 | 1758638 | 0.0465 |
| STX16 | 20q13.32 | 8675 | 221500_s_at | 0.0039 | 1688537 | 0.0063 |
| C20orf43 | 20q13.31 | 51507 | 217737_x_at | 0.0191 | 1667932 | 0.0148 |
| RAB22A | 20q13.32 | 57403 | 218360_at | 0.001 | 1645691 | 0.0077 |
| HDAC1 | 1p34 | 3065 | 201209_at | −0.0382 | 1656045 | −0.0266 |
| BSDC1 | 1p35.1 | 55108 | 218004_at | −0.0196 | 1677842 | −0.0266 |
| C1orf9 | 1q24 | 51430 | 203429_s_at | 0.0429 | 1707822 | 0.0024 |
| COX5B | 2cen-q13 | 1329 | 211025_x_at | 0.0145 | 1705118 | 0.0018 |
| EIF5B | 2q11.2 | 9669 | 201025_at | 0.0441 | 1728008 | 0.0076 |
| DDX18 | 2q14.1 | 8886 | 208896_at | 0.0143 | 1696503 | 0.0061 |
| TSN | 2q21.1 | 7247 | 201513_at | 0.0416 | 1673463 | 0.0455 |
| p20 | 2q21.1 | 130074 | 212017_at | 0.0308 | 1718104 | 0.011 |
| METTL5 | 2q31.1 | 29081 | 221570_s_at | 0.0397 | 1652493 | 0.0045 |
| MGAT1 | 5q35 | 4245 | 201126_s_at | 0.0156 | 1683255 | 0.0185 |
| TUBB2A | 6p25 | 7280 | 204141_at | 0.0152 | 1713325 | 0.0487 |
| RWDD1 | 6q13-q22.33 | 51389 | 219598_s_at | 0.0158 | 1750430 | 0.0311 |
| PGM3 | 6q14.1-q15 | 5238 | 210041_s_at | 0.003 | 1724282 | 0.0413 |
| FOXO3 | 6q21 | 2309 | 204131_s_at | 0.048 | 1645067 | 0.0459 |
| CDC40 | 6q21 | 51362 | 203376_at | 0.0037 | 1711755 | 0.0306 |
| REV3L | 6q21 | 5980 | 208070_s_at | 0.004 | 1667275 | 0.0468 |
| HDAC2 | 6q21 | 3066 | 201833_at | 0.0362 | 1645015 | 0.0007 |
| TSPYL4 | 6q22.1 | 23270 | 212928_at | 0.0146 | 1669819 | 0.0098 |
| C6orf60 | 6q22.31 | 79632 | 220150_s_at | 0.0259 | 1694717 | 0.0129 |
| ASF1A | 6q22.31 | 25842 | 203427_at | 0.0148 | 1740438 | 0.0168 |
| MED23 | 6q22.33-q24.1 | 9439 | 218846_at | 0.0453 | 1661877 | 0.0186 |
| TSPYL1 | 6q22-q23 | 7259 | 221493_at | 0.0155 | 1758155 | 0.0144 |
| ACTR10 | 14q23.1 | 55860 | 222230_s_at | 0.0011 | 1741052 | 0.0343 |
| KIAA0247 | 14q24.1 | 9766 | 202181_at | 0.0128 | 1702018 | 0.0005 |
| RARA | 17q21 | 5914 | 203749_s_at | 0.0474 | 1731414 | 0.0281 |
| KRT10 | 17q21 | 3858 | 213287_s_at | 0.0309 | 1735532 | 0.0251 |
| RIOK3 | 18q11.2 | 8780 | 202130_at | 0.0134 | 1740064 | 0.0024 |
| IMPACT | 18q11.2-q12.1 | 55364 | 218637_at | 0.016 | 1684789 | 0.017 |

TABLE 6B
Description of the 81 genes used as the CNS (continued)

| | | gene | | | |
|---|---|---|---|---|---|
| | | expression | diploid | | |
| | gain or | & copy | copy | copy | |
| gene | loss | number | number | number | |
| symbol | (1 = gain; −1 = loss) | correlation | estimate | cutoff | description |
| SMC4 | 1 | 0.519 | 2.176 | 3.676 | SMC4 structural maintenance of chromosomes |
| | | | | | 4-like 1 (yeast) |
| PDCD10 | 1 | 0.756 | 2.108 | 3.608 | programmed cell death 10 |
| PREP | 1 | 0.722 | 2.133 | 3.633 | prolyl endopeptidase |
| CBX3 | 1 | 0.585 | 2.187 | 3.687 | chromobox homolog 3 (HP1 gamma homolog, |
| | | | | | Drosophila) |
| NUP205 | 1 | 0.576 | 2.153 | 3.653 | nucleoporin 205 kDa |
| TCEB1 | 1 | 0.653 | 2.348 | 3.848 | transcription elongation factor B (SIII), |
| | | | | | polypeptide 1 (15 kDa, elongin C) |
| TERF1 | 1 | 0.801 | 2.729 | 4.229 | telomeric repeat binding factor (NIMA-interacting) 1 |
| TPD52 | 1 | 0.624 | 1.904 | 3.404 | tumor protein D52 |
| GGH | 1 | 0.528 | 2.011 | 3.511 | gamma-glutamyl hydrolase (conjugase, |
| | | | | | folylpolygammaglutamyl hydrolase) |
| TRAM1 | 1 | 0.618 | 2.211 | 3.711 | translocation associated membrane protein 1 |
| ZBTB10 | 1 | 0.674 | 2.027 | 3.527 | zinc finger and BTB domain containing 10 |
| YTHDF3 | 1 | 0.62 | 1.922 | 3.422 | YTH domain family, member 3 |
| EIF3E | 1 | 0.544 | 2.106 | 3.606 | eukaryotic translation initiation factor 3, subunit 6 |
| | | | | | 48 kDa |
| POLR2K | 1 | 0.694 | 2.216 | 3.716 | polymerase (RNA) II (DNA directed) polypeptide |
| | | | | | K, 7.0 kDa |
| RPL30 | 1 | 0.698 | 2.227 | 3.727 | ribosomal protein L30 |
| CCNE2 | 1 | 0.527 | 2.241 | 3.741 | cyclin E2 |
| RAD54B | 1 | 0.692 | 1.954 | 3.454 | RAD54 homolog B (S. cerevisiae) |
| MTERFD1 | 1 | 0.788 | 2.45 | 3.95 | MTERF domain containing 1 |
| ENY2 | 1 | 0.775 | 2.009 | 3.509 | enhancer of yellow 2 homolog (Drosophila) |
| DPY19L4 | 1 | 0.58 | 1.979 | 3.479 | dpy-19-like 4 (C. elegans) |
| ZNF623 | 1 | 0.618 | 1.837 | 3.337 | zinc finger protein 623 |
| SCRIB | 1 | 0.735 | 1.837 | 3.337 | scribbled homolog (Drosophila) |
| SLC39A4 | 1 | 0.64 | 1.837 | 3.337 | solute carrier family 39 (zinc transporter), |
| | | | | | member 4 |
| ATP6V1G1 | 1 | 0.518 | 2.214 | 3.714 | ATPase, H+ transporting, lysosomal 13 kDa, V1 |
| | | | | | subunit G1 |
| TCTN3 | −1 | 0.577 | 2.288 | 1.788 | chromosome 10 open reading frame 61 |
| PSMA6 | 1 | 0.616 | 2.226 | 3.726 | proteasome (prosome, macropain) subunit, alpha |
| | | | | | type, 6 |
| STRN3 | 1 | 0.503 | 2.122 | 3.622 | striatin, calmodulin binding protein 3 |
| CLTC | 1 | 0.883 | 1.939 | 3.439 | clathrin, heavy polypeptide (Hc) |
| TRIM37 | 1 | 0.781 | 2.555 | 4.055 | tripartite motif-containing 37 |
| NME1 | 1 | 0.812 | 1.805 | 3.305 | non-metastatic cells 1, protein (NM23A) |
| | | | | | expressed in |
| NME2 | 1 | 0.743 | 1.624 | 3.124 | non-metastatic cells 2, protein (NM23B) |
| | | | | | expressed in |
| RPS6KB1 | 1 | 0.758 | 2.027 | 3.527 | ribosomal protein S6 kinase, 70 kDa, polypeptide |
| | | | | | 1 |
| PPM1D | 1 | 0.85 | 2.049 | 3.549 | protein phosphatase 1D magnesium-dependent, |
| | | | | | delta isoform |
| MED13 | 1 | 0.778 | 2.164 | 3.664 | thyroid hormone receptor associated protein 1 |
| SLC35B1 | 1 | 0.78 | 2.318 | 3.818 | solute carrier family 35, member B1 |
| APPBP2 | 1 | 0.857 | 2.063 | 3.563 | amyloid beta precursor protein (cytoplasmic tail) |
| | | | | | binding protein 2 |
| MKS1 | 1 | 0.555 | 2.13 | 3.63 | Meckel syndrome, type 1 |
| C17orf71 | 1 | 0.86 | 2.555 | 4.055 | chromosome 17 open reading frame 71 |
| HEATR6 | 1 | 0.782 | 2.104 | 3.604 | — |
| TMEM49 | 1 | 0.706 | 1.913 | 3.413 | transmembrane protein 49 |
| USP32 | 1 | 0.812 | 2.146 | 3.646 | ubiquitin specific peptidase 32 |
| ANKRD40 | 1 | 0.62 | 2.157 | 3.657 | ankyrin repeat domain 40 |
| NME1- | 1 | 0.77 | 1.805 | 3.305 | — |
| NME2 | | | | | |
| ZNF264 | 1 | 0.557 | 1.661 | 3.161 | zinc finger protein 264 |
| ZNF304 | 1 | 0.78 | 1.649 | 3.149 | zinc finger protein 304 |
| ATP5E | 1 | 0.514 | 1.99 | 3.49 | ATP synthase, H+ transporting, mitochondrial F1 |
| | | | | | complex, epsilon subunit |
| CSTF1 | 1 | 0.526 | 1.866 | 3.366 | cleavage stimulation factor, 3′ pre-RNA, subunit |
| | | | | | 1, 50 kDa |
| PPP1R3D | 1 | 0.601 | 2.231 | 3.731 | protein phosphatase 1, regulatory subunit 3D |
| AURKA | 1 | 0.577 | 1.866 | 3.366 | aurora kinase A |
| RAE1 | 1 | 0.676 | 2.475 | 3.975 | RAE1 RNA export 1 homolog (S. pombe) |
| STX16 | 1 | 0.61 | 2.179 | 3.679 | syntaxin 16 |
| C20orf43 | 1 | 0.509 | 1.912 | 3.412 | chromosome 20 open reading frame 43 |
| RAB22A | 1 | 0.801 | 2.52 | 4.02 | RAB22A, member RAS oncogene family |
| HDAC1 | −1 | 0.551 | 2.329 | 1.829 | histone deacetylase 1 |
| BSDC1 | −1 | 0.616 | 2.259 | 1.759 | BSD domain containing 1 |
| C1orf9 | 1 | 0.532 | 2.448 | 3.948 | chromosome 1 open reading frame 9 |
| COX5B | 1 | 0.739 | 1.846 | 3.346 | cytochrome c oxidase subunit Vb |
| EIF5B | 1 | 0.618 | 1.706 | 3.206 | eukaryotic translation initiation factor 5B |
| DDX18 | 1 | 0.581 | 2.186 | 3.686 | DEAD (Asp-Glu-Ala-Asp) box polypeptide 18 |
| TSN | 1 | 0.626 | 2.308 | 3.808 | translin |
| p20 | 1 | 0.537 | 1.701 | 3.201 | LOC130074 |
| METTL5 | 1 | 0.509 | 2.158 | 3.658 | methyltransferase like 5 |
| MGAT1 | 1 | 0.848 | 2.435 | 3.935 | mannosyl (alpha-1,3-)-glycoprotein beta-1,2-N- |
| | | | | | acetylglucosaminyltransferase |
| TUBB2A | 1 | 0.563 | 2.221 | 3.721 | tubulin, beta 2A |
| RWDD1 | 1 | 0.655 | 1.996 | 3.496 | RWD domain containing 1 |
| PGM3 | 1 | 0.787 | 2.052 | 3.552 | phosphoglucomutase 3 |
| FOXO3 | 1 | 0.823 | 2.259 | 3.759 | forkhead box O3 |
| CDC40 | 1 | 0.715 | 2.261 | 3.761 | cell division cycle 40 homolog (S. cerevisiae) |
| REV3L | 1 | 0.614 | 1.9 | 3.4 | REV3-like, catalytic subunit of DNA polymerase |
| | | | | | zeta (yeast) |
| HDAC2 | 1 | 0.639 | 2.034 | 3.534 | histone deacetylase 2 |
| TSPYL4 | 1 | 0.501 | 1.863 | 3.363 | TSPY-like 4 |
| C6orf60 | 1 | 0.531 | 1.916 | 3.416 | chromosome 6 open reading frame 60 |
| ASF1A | 1 | 0.669 | 1.821 | 3.321 | ASF1 anti-silencing function 1 homolog A (S. |
| | | | | | cerevisiae) |
| MED23 | 1 | 0.564 | 2.03 | 3.53 | mediator complex subunit 23 |
| TSPYL1 | 1 | 0.529 | 1.916 | 3.416 | TSPY-like 1 |
| ACTR10 | 1 | 0.635 | 1.965 | 3.465 | actin-related protein 10 homolog (S. cerevisiae) |
| KIAA0247 | 1 | 0.573 | 1.913 | 3.413 | KIAA0247 |
| RARA | 1 | 0.685 | 2.08 | 3.58 | retinoic acid receptor, alpha |
| KRT10 | 1 | 0.777 | 2.085 | 3.585 | keratin 1 |
| RIOK3 | 1 | 0.594 | 2.021 | 3.521 | RIO kinase 3 (yeast) |
| IMPACT | 1 | 0.556 | 2.242 | 3.742 | Impact homolog (mouse) |

The top 53 genes are from ER-positive tumors, the bottom 28 are from ER-negative tumors.

TABLE 7
Prognostic chromosome regions in ER-positive tumors

| | start | end | copy number change |
|---|---|---|---|
| chromosome | (base) | (base) | (1 = gains; −1 = loss) |
| 1 | 10678225 | 18511423 | −1 |
| 1 | 28955687 | 32872286 | −1 |
| 1 | 83788073 | 104676601 | −1 |
| 2 | 9818363 | 14413615 | −1 |
| 2 | 24752932 | 25901745 | −1 |
| 2 | 95284610 | 95979338 | 1 |
| 2 | 130443728 | 136187793 | −1 |
| 3 | 48603 | 5655734 | 1 |
| 3 | 8147792 | 11885879 | 1 |
| 3 | 49266749 | 50512778 | −1 |
| 3 | 151441127 | 172623620 | 1 |
| 3 | 173869649 | 180099794 | 1 |
| 4 | 103115 | 10491185 | −1 |
| 4 | 35641248 | 38921691 | −1 |
| 6 | 104481650 | 110713418 | 1 |
| 7 | 250149 | 43854476 | 1 |
| 7 | 49374011 | 54893546 | 1 |
| 7 | 132167036 | 138790478 | 1 |
| 8 | 47365080 | 48965918 | 1 |
| 8 | 56155338 | 90048318 | 1 |
| 8 | 91075378 | 92102438 | 1 |
| 8 | 94156558 | 113670698 | 1 |
| 8 | 143455438 | 146023088 | 1 |
| 9 | 42004193 | 42930351 | 1 |
| 9 | 68229855 | 94387165 | 1 |
| 9 | 97218677 | 122702285 | 1 |
| 10 | 25372233 | 28876308 | −1 |
| 10 | 47564708 | 48732733 | −1 |
| 10 | 49900758 | 51068783 | −1 |
| 10 | 82605458 | 134582570 | −1 |
| 11 | 802188 | 16154613 | −1 |
| 11 | 68966955 | 73879731 | 1 |
| 11 | 98443611 | 133447140 | −1 |
| 12 | 42668236 | 46370371 | 1 |
| 12 | 69817226 | 85859811 | 1 |
| 12 | 87093856 | 88327901 | 1 |
| 14 | 22535406 | 36835923 | 1 |
| 14 | 44636205 | 49836393 | 1 |
| 14 | 53736534 | 60236769 | 1 |
| 14 | 83637615 | 90137850 | 1 |
| 16 | 32070490 | 33891366 | −1 |
| 17 | 42580727 | 60216632 | 1 |
| 18 | 25801802 | 75535109 | −1 |
| 19 | 61179186 | 63432439 | 1 |
| 20 | 47547185 | 60690155 | 1 |

TABLE 8
Prognostic chromosome regions in ER-negative tumors

| | start | end | copy number change |
|---|---|---|---|
| chromosome | (base) | (base) | (1 = gains; −1 = loss) |
| 1 | 21122489 | 34177819 | −1 |
| 1 | 115120865 | 120839024 | −1 |
| 1 | 167342185 | 175175383 | 1 |
| 1 | 224785637 | 226091170 | 1 |
| 2 | 61514948 | 73003078 | −1 |
| 2 | 82193582 | 88993415 | −1 |
| 2 | 95284610 | 101723403 | 1 |
| 2 | 108616281 | 156866427 | 1 |
| 2 | 164908118 | 172949809 | 1 |
| 2 | 192479630 | 195926069 | 1 |
| 2 | 215455890 | 228092833 | −1 |
| 2 | 230390459 | 239580963 | −1 |
| 2 | 240729776 | 241304182 | −1 |
| 3 | 31822343 | 44282633 | −1 |
| 3 | 48020720 | 50512778 | −1 |
| 3 | 95122010 | 97861880 | 1 |
| 3 | 151441127 | 157671272 | 1 |
| 4 | 30173843 | 31814064 | 1 |
| 4 | 55323906 | 61884792 | −1 |
| 4 | 71726121 | 77193526 | −1 |
| 5 | 18740255 | 19799220 | −1 |
| 5 | 30388870 | 40978520 | −1 |
| 5 | 47332310 | 48391275 | −1 |
| 5 | 170172250 | 176526040 | 1 |
| 5 | 177585005 | 180232417 | 1 |
| 6 | 1657478 | 6850618 | 1 |
| 6 | 62010774 | 66052414 | 1 |
| 6 | 76438694 | 97211254 | 1 |
| 6 | 107597534 | 123176954 | 1 |
| 6 | 127331466 | 132524606 | 1 |
| 7 | 91322477 | 93530291 | −1 |
| 7 | 99049826 | 100153733 | −1 |
| 7 | 106777175 | 114504524 | −1 |
| 7 | 136030710 | 139342431 | −1 |
| 9 | 55453875 | 66072045 | −1 |
| 10 | 42236621 | 51068783 | −1 |
| 11 | 34577523 | 45631269 | 1 |
| 11 | 54916541 | 73879731 | −1 |
| 11 | 93530835 | 94759029 | −1 |
| 12 | 36498011 | 45136326 | −1 |
| 12 | 58093798 | 62412956 | 1 |
| 12 | 130285431 | 131519476 | 1 |
| 14 | 35535876 | 38135970 | 1 |
| 14 | 54386557 | 71937192 | 1 |
| 14 | 103138320 | 105088390 | −1 |
| 16 | 17503482 | 32070490 | −1 |
| 16 | 73950638 | 87607208 | −1 |
| 17 | 34742547 | 40621182 | 1 |
| 18 | 16836580 | 28942853 | 1 |
| 18 | 36271972 | 37318989 | 1 |
| 19 | 36393397 | 52166172 | −1 |
| 20 | 49007515 | 61420320 | −1 |
| 21 | 41980980 | 45609552 | −1 |
| 23 | 677050 | 24691270 | −1 |
| 23 | 34296958 | 56710230 | −1 |
| 23 | 130353838 | 154368058 | −1 |

What is claimed is:

1. A method of defining chromosome regions of prognostic value comprising the step of summarizing the significance of all SNPs in a predetermined section of a chromosome to define chromosome regions of prognostic value.

2. The method according to claim 1 wherein the step of summarizing is done by determining the P value of Cox proportion hazard regression of each SNP in the region and summarizing the combined P values.

3. The method according to claim 1 further comprising the step of correlating the SNP copy numbers with the levels of expression of genes located within the predetermined chromosome section.

4. The method according to claim 1, further comprising the step of developing a treatment regiment based on the combined P values.

5. A method for providing a prognosis for human breast cancer comprising the steps of
obtaining a DNA sample from a human;
examining the DNA sample for a single nucleotide polymorphism in at least gene selected from the group consisting of SMC4, PDCD10, PREP, CBX3, NUP205, TCEB1, TERF1, TPD52, GGH, TRAM1, ZBTB10, YTHDF3, EIF3E, POLR2K, RPL30, CCNE2, RAD54B, MTERFD1, ENY2, DPY19L4, ZNF623, SCRIB, SLC39A4, ATP6V1G1, TCTN3, PSMA6, STRN3, CLTC, TRIM37, NME1, NME2, RPS6KB1, PPM1D, MED13, SLC35B1, APPBP2, MKS1, C17orf71, HEATR6, TMEM49, USP32, ANKRD40, NME1-NME2, ZNF264, ZNF304, ATP5E, CSTF1, PPP1R3D, AURKA, RAE1, STX16, C20orf43, RAB22A, HDAC1, BSDC1, C1orf9, COX5B, EIF5B, DDX18, TSN, p20, METTL5, MGAT1, TUBB2A, RWDD1, PGM3, FOXO3, CDC40, REV3L, HDAC2, TSPYL4, C6orf60, ASF1A, MED23, TSPYL1, ACTR10, KIAA0247, RARA, KRT10, RIOK3, IMPACT, and combinations thereof;
providing a prognosis for human breast cancer based on the results of the step of examining the DNA sample.

6. The method as recited in claim 5, further comprising the step of obtaining a breast tumor sample from the human.

7. The method as recited in claim 6, further comprising the step of determining whether the tumor sample is estrogen-receptor positive or estrogen-receptor negative.

8. The method as recited in claim 7, wherein the tumor sample is determined to be estrogen-receptor positive and the single nucleotide polymorphism is determined to be a loss in TCTN3.

9. The method as recited in claim 7, wherein the tumor sample is determined to be estrogen-receptor negative and the single nucleotide polymorphism is determined to be a loss in HDAC1, BSDC1, or a combination thereof.

10. A method for providing a prognosis for human breast cancer comprising the steps of
obtaining a DNA sample from a human;
examining the DNA sample for a single nucleotide polymorphism on at least one chromosome selected from the group consisting of chromosome numbers 1, 2, 3, 4, 5, 6, 7, 9, 10, 11, 12, 14, 16, 17, 18, 19, 20, 21, 23, and combinations thereof, wherein the single nucleotide polymorphism occurs between the corresponding starting base and ending base recited in Tables 7 and 8;
providing a prognosis for human breast cancer based on the results of the step of examining the DNA sample.

11. The method as recited in claim 10, further comprising the step of obtaining a breast tumor sample from the human.

12. The method as recited in claim 11, further comprising the step of determining whether the tumor sample is estrogen-receptor positive or estrogen-receptor negative.

13. The method as recited in claim 12, wherein the tumor sample is determined to be estrogen-receptor positive and the single nucleotide polymorphism occurs between the corresponding starting base and ending base recited in Table 7.

14. The method as recited in claim 12, wherein the tumor sample is determined to be estrogen-receptor negative and the single nucleotide polymorphism occurs between the corresponding starting base and ending base recited in Table 8.
